(19) United States
(12) Patent Application Publication
(10) Pub. No.: US 2004/0052835 A1
(43) Pub. Date: Mar. 18, 2004

(54) Matrix controlled transdermal system for stabile derivatives of ace inhibitors

(76) Inventors: Karin Klokkers, Feldkirchen-Westerham (DE); Kai-Thomas Kramer, Bad Tolz (DE); Wilfried Fischer, Miesbach (DE); Anna Sendl-Lang, Nouriad (DE)

Correspondence Address: Vincent M. Fazzari, Cohen Pontani Lieberman & Pavane, 551 Fifth Avenue Suite 1210, New York, NY, 10176, US

(21) Appl. No.: 10/019,121

(22) PCT Filed: Jul. 21, 2001
(86) PCT No.: PCT/EP01/08071

(30) Foreign Application Priority Data
Jul. 12, 2000 (DE) 100 33 855.0

(51) Int. Cl. A61K 31/401; A61K 9/70

(52) U.S. Cl. 424/449; 514/423

(57) ABSTRACT

The invention relates to a matrix-controlled transdermal therapeutic system comprising (i) an active-ingredient-impermeable cover layer, (ii) a self-adhesive matrix layer, or a plurality of matrix layers of which at least the matrix layer exposed while applying the system is self-adhesive, or one or more matrix layers whose surface remote from the cover layer and intended for adhesion at the application site is coated with an adhesive, the matrix layers comprising at least one ACE inhibitor (angiotensin converting enzyme inhibitor), the metabolite of which is a dicarboxylic acid and which is selected from the following group:

diester,

di-salt obtainable with base(s) and

mono-salt obtainable with acid(s),

and (iii) a removable protective layer.

The invention relates to a stable, active-ingredient-containing transdermal therapeutic system for application of stable derivatives of those ACE inhibitors whose metabolites are in the form of a dicarboxylic acid. Stable derivatives of those ACE inhibitors are obtained by salt formation from, or esterification of, the dicarboxylic acids.

Long-term therapy of hypertension using angiotensin converting enzyme inhibitors (ACE inhibitors) is gaining in importance.

ACE inhibitors are known for their reliable efficacy, together with good tolerability.

Hitherto, only oral dosage forms of ACE inhibitors, such as tablets or capsules, have been commercially available. In the case of oral dosage forms it is disadvantageous that the patient has to swallow at least one tablet or capsule every day and that the blood plasma level is always subject to certain fluctuations. Using oral dosage forms it is virtually impossible to ensure a steady plasma level.

Transdermal administration, on the other hand, has a number of advantages for ACE inhibitors:

the skin is accessible without restriction;

there is no change of medium, such as that occurring in the case of peroral administration;

handling is simple and convenient;

a single administration, instead of multiple daily administrations, is usually sufficient;

patient compliance is significantly better;

continuous long-term therapy is possible;

the active ingredient is released almost according to zero-order kinetics;

therapy can be interrupted more rapidly;

a constant plasma level is ensured for a relatively long period;

a plasma level that initially is too high, such as that occurring in the case of intravenous administration, is avoided;

and

by virtue of avoidance of the first-pass effect, a lower dose is in some instances required than in the case of oral administration, which results in a lower rate of side-effects and a lower risk of an over- or under-dose.

W0-A1-93/23019 discloses a transdermal reservoir system containing an ACE inhibitor and

a) an impermeable covering layer (backing layer),

b) a layer-like element having a hollow space,

c) a means controlling active ingredient release (claim 1) and

d) a removable cover layer (release liner) based on paper (page 12 line 7/8).

Transdermal systems containing an ACE inhibitor are further described in EP-A 0 439 430 (reservoir TTS) and EP-A-0 468 875 (matrix TTS), wherein according to EP-A-0 468 875 silicone elastomers are used as matrix material. EP-A452 837 describes a matrix for patches comprising, inter alia, ACE inhibitors as active ingredients, although specific mention is made of delapril hydrochloride, enalapril maleate, captopril, alacepril and (R)-3-[(S)-1-carboxy-5-(4-piperidyl)-pentyl]-amino-4-oxo-2,3,4,5-tetrahydro-1,5-benzothiazepine-5-acetic acid as possible ACE inhibitors. WO 96/29999 describes a TTS having a matrix based on polyisobutylene or butyl rubber containing trandolapril and/or ramipril.

It has now been found that, for a matrix TTS of certain non-stabilised ACE inhibitors, the stability of the active ingredient in the patch does not meet requirements. Decomposition of the ACE inhibitor in the matrix occurs to such a great extent that, even after a short period of storage, the content of decomposition products is so high that the tolerance limit for degradation products is far exceeded. In addition, it is not possible, using non-stabilised ACE inhibitors, to achieve adequate permeation through skin in vitro.

The problem of the present invention is to provide a matrix TTS containing a stable derivative of ACE inhibitors, the stability of which derivative with respect to degradation of the active ingredient satisfies the legal requirements and the in vitro skin p rmeation of which derivative is greatly increased. The active ingredient content should be stable over a relatively long period and be subject to virtually no decomposition processes. The adhesive strength of the matrix patch should be sufficient for a period of wear lasting at least 3 days.

It has now been found, surprisingly, that the salts of the active metabolites (=dicarboxylic acids) of ACE inhibitors, which are formed by reaction of the dicarboxylic acid with strong acids (1:1) or bases (1:2), are substantially stable with respect to decomposition, on the one hand, and exhibit outstanding skin permeation, on the other hand, it being possible for the salts of the active metabolites to be formed in situ in the matrix layer.

Stabilisation of the ACE inhibitors can, surprisingly, also be achieved by diesterification of their metabolites. As a result of the associated increase in the lipophilicity of the ACE inhibitor, it is possible to obtain excellent skin permeation.

In some cases, the stable derivatives of ACE inhibitors are so hygroscopic that the patch comes away from the skin after only a short time. That problem has been solved by additionally applying an “overtape” over the actual matrix patch, the latter consisting of a cover layer impermeable to the active ingredient, an active-ingredient-containing, self-adhesive matrix and a removable protective layer. The “overtape” can extend beyond the actual matrix patch on all sides. An “overtape” is understood to mean a composite of a cover layer and an adhesive layer.

The problem underlying the invention is accordingly solved by a matrix-controlled transdermal therapeutic system containing at least one stable derivative of an ACE inhibitor. The adhesive strength of the patch and its wearability characteristics can be significantly improved, where necessary, by the application of an “overtape”.

The transdermal therapeutic system according to the invention may comprise a cover layer (5) impermeable to the active ingredient, one or more self-adhesive matrix layer(s) (6) comprising the active ingredient and/or optional permeation enhancers or one or more matrix layer(s) (9) coated with an adhesive (8), and a removable protective layer (7). In the case of hygroscopic stabilised ACE inhibitors, an “overtape” (1), composed of a composite of a cover layer (3) and an adhesive layer (4), is used. The “overtape” can extend b yond the rest of the system (2) on all sides.

In the transdermal therapeutic system according to the invention there may be used at least one stable salt of the active metabolites (=dicarboxylic acids) of an ACE inhibitor, which salt is based on reaction of an acid or alkaline compound with the dicarboxylic acid. Acid compounds that come into consideration are: inorganic acids, for example hydrochloric acid, hydrobromic acid, hydriodic acid, nitric acid, sulphuric acid, phosphoric acid, organic carboxylic acids, for example salicylic acid, maleic acid, adipic acid, sorbic acid, malonic acid, 1,4-butanedioic acid, malic acid, pivalic acid, succinic acid, nicotinic acid, isonicotinic acid, furan-2-carboxylic acid, dichloroacetic acid, benzoic acid, fatty acids, for example lauric acid, myristic acid, oleic acid, aliphatic sulphonic acids, for example methane-, ethane-, propane-, isopropane-, butane-, isobutane-, pentane-, isopentane-, hexane-, heptane-, octane-, nonane-, decane-, undecane-, dodecane-sulphonic acid or aromatic sulphonic acids, for example toluene- or benzene-sulphonic acid, especially methane-, toluene- or benzene-sulphonic acid. The acid preferably used is methane-sulphonic acid.

Alkaline compounds that come into consideration are: sodium hydroxide, potassium hydroxide, magnesium hydroxide, calcium hydroxide, aluminium hydroxide, alkaline ammonium salts, organic amines, for example ethylenediamine, ethylamine, diethylamine, dipropylamine, diisopropylamine, tripropylamine, trihexylamine, tridodecylamine, dicyclohexylamine, ethanolamine, diethanolamine, triethanolamine, triisopropanolamine, 1-amino-2-propanol, 2-amino-2-methyl-1-propanol, oleylamine, heterocyclic amines, for example N-methylpiperazine or 1-(2-hydroxyethyl)pyrrolidine. The base preferably used is sodium hydroxide.

The stable salts of the active metabolites (=dicarboxylic acids) of the ACE inhibitors can be formed in situ in the matrix by incorporating the appropriate alkaline or acid compounds and the dicarboxylic acids together in the matrix.

The stable salts of the metabolites of the ACE inhibitors can, however, also be introduced into the matrix directly.

In an alternative embodiment of the transdermal therapeutic system according to the invention, at least one stable diester of an ACE inhibitor metabolite may be used. As the alkyl radical of the ester there come into consideration the methyl, ethyl, propyl, isopropyl, butyl, tert-butyl, pentyl, hexyl, heptyl, octyl, nonane and decane groups and isomers thereof. The ethyl est r is preferred.

The transdermal therapeutic system according to the inv ntion may compris, as active ingredient, the stabilised forms of those ACE inhibitors whose active metabolites are in the form of a dicarboxylic acid. Examples are imidapril, fosinopril, moexipril, perindopril, ramipril, spirapril, cilazapril, benazepril and/or trandolapril. Preferably, the monosulphonic acid salts, the disodium salts and the diesters of trandolaprilat and/or ramiprilat are used as active ingredient components.

The content of ACE inhibitors may be from 2 to 25% by weight, especially from 10 to 15% by weight, based on the matrix weight.

The content of acid or base is equimolar to the metabolite (=dicarboxylic acid) of the ACE inhibitor, that of base being twice as great, and accordingly is dependent upon the molecular weight of the dicarboxylic acid.

As the impermeable cover layer there come into consideration films of acetal, acrylate, acrylonitrile/butadiene/styrene, acrylonitrile (methyl methacrylate) copolymer, acrylonitrile copolymer, ethylene ethyl acrylate, ethylene methyl acrylate, ethylene vinyl acetate, ethylene vinyl acetate copolymer, ethylene vinyl alcohol polymer, ionomers, nylon (polyamide), nylon (polyamide) copolymer, polybutylene, polycarbonate, polyester, polyethylene terephthalate, thermoplastic polyester copolymer, polyethylene copolymer (high density), polyethylene (high-molecular-weight, high-density), polyethylene (intermediate-molecular-weight, high-density), polyethylene (linear low density), polyethylene (low density), polyethylene (medium density), polyethylene oxide, polyimide, polypropylene, polypropylene (coated), polypropylene (oriented), polystyrene, polyurethane, polyvinyl acetate, polyvinyl chloride, polyvinylidene chloride and/or styrene/acrylonitrile, which may if necessary be metal-coated or pigmented. As the active-ingredient-impermeable cover layer, polyurethane is preferred.

As the cover layer for the overtape there come into consideration micro-perforated films of the above-mentioned materials and membranes of polyurethane, polyethylene, polypropylene, polyester and coextruded materials of ethyl vinyl alcohol/polyethylene or polyethylene/polypropylen. As the cover lay r for the overtape, polyur than st rs and polyurethane ethers ar preferred.

For the adhesive layer, especially of the overtape, there may be selected a pressure-sensitive adhesive, for example based on polyurethane, on polyisobutylene, on polyvinyl ether, on polyacrylate or a mixture thereof. Preferably, adhesives based on acrylate and polyisobutylene are used.

The adhesives based on polyacrylate may be any homopolymer, copolymer or terpolymer, consisting of various acrylic acid derivatives.

The polyacrylates may accordingly be polymers of one or more monomers of acrylic acids and other copolymerisable monomers. In addition, the polyacrylates may include copolymers of alkyl acrylates and/or alkyl methacrylates and/or copolymerisable secondary monomers or monomers containing functional groups. By varying the amount of each monomer species added it is possible to vary the cohesive properties of the acrylate polymers resulting therefrom. In general, the acrylate polymer consists of at least 50% by weight of an acrylate, methacrylate, alkyl acrylate or alkyl methacrylate monomer, from 0 to 20% of a functional monomer copolymerisable with acrylate and from 0 to 50% of another monomer.

Hereinafter there are mentioned various acrylate monomers, for example acrylic acid, methacrylic acid, butyl acrylate, butyl methacrylate, hexyl acrylate, hexyl methacrylate, isooctyl acrylate, isooctyl methacrylate, glycidyl methacrylate, 2-hydroxyethyl acrylate, methyl acrylate, methyl methacrylate, 2-ethylhexyl acrylate, 2-ethylhexyl methacrylate, decyl acrylate, decyl methacrylate, dodecyl acrylate, dodecyl methacrylate, tridecyl acrylate and tridecyl methacrylate, which can be polymerised alone or in admixture.

In addition, functional monomers that are copolymerisable with the above-mentioned acrylates, for example acrylic acid, methacrylic acid, maleic acid, maleic anhydride, hydroxyethyl acrylate, vinyl acetate, hydroxypropyl acrylate, acrylamide, dimethyl acrylamide, acrylonitrile, dimethyl aminoethyl acrylate, dimethyl aminoethyl methacrylate, tert-butyl aminoethyl acrylate, tert-butyl aminoethyl methacrylate, methoxyethyl acrylate, methoxyethyl methacrylate, can be used for copolymerisation.

Further details and examples of pressure-sensitive acrylates that are suitable for the invention are describ d in Satas' Handbook of Pressure Sensitive Adhesive Technology “Acrylic Adhesives”, 2nd End., pp. 396456 (D. Satas, Ed.), Van Nostrand Reinhold, New York (1989).

The adhesive content of the self-adhesive matrix may be from 50 to 90% by weight, especially from 70 to 80% by weight, based on the matrix weight.

For the matrix there are used the medically customary matrix-formers, for example polyacrylate, polyisobutylene, natural rubber, natural-rubber-like synthetic homo-, co- or block polymers, styrene/butadiene copolymer or a mixture thereof, as provided in the prior art. Preferably, a self-adhesive matrix of polyacrylate and/or polyisobutylene is used, the matrix-former and the adhesive then being identical.

For the removable protective layer there come into consideration polyester, polyethylene, polypropylene, polysiloxane, polyacrylate, ethylene vinyl acetate, polyurethane, polyisobutene or paper, usually coated with silicone and/or polyethylene, or a mixture thereof.

As permeation enhancers there may be used, where appropriate, saturated and/or unsaturated fatty alcohols in each case containing from 8 to 18 C atoms; tea tree oil; saturated and/or unsaturated cyclic ketones; alkyl methyl sulphoxides; saturated and/or unsaturated fatty acids in each case containing from 8 to 18 C atoms; esters and salts thereof; natural vitamin E; synthetic vitamin E and/or derivatives of vitamin E; sorbitan fatty acid esters and ethoxylated sorbitan fatty acid esters; Azone (laurocapram); 1-alkyl-pyrrolidone; block copolymers of polyethylene glycol and dimethylsiloxane with a cationic group at one end; polyoxyethylene 10-stearyl ether; mixture of polyoxyethylene 10-stearyl ether and glyceryl dilaurate; dodecyl 2-(N,N-dimethylamino)-propanol tetradecanoate and/or dodecyl 2-(N,N-dimethylamino)-propionate; N-acetyl prolinate ester having >8 C atoms; non-ionic surfactants, for example lauryl ethers, esters of polyoxyethylene; dimethyl(arylimino)sulphuran; mixture of oleic acid analogues and propylene glycol; mixture of Padimate O, octyl salicylate, isopropyl myristate, isopropyl palmitate, octyl methoxycinnimate, laurocapram; highly disperse silicon dioxide (Aerosil®); polyoxyethylene 7-glycerol monococoate (Cetiol® HE); 2-octyidodecanol (Eutanol® G) or a mixture of various individual components. In the transdermal therapeutic system according to the invention, highly disperse silicon dioxide (Aerosil®) and/or polyoxyethylene 7-glycerol monococoate (Cetiol® HE) and/or 2-octyldodecanol (Eutanol® G) are preferred as optional permeation enhancer(s).

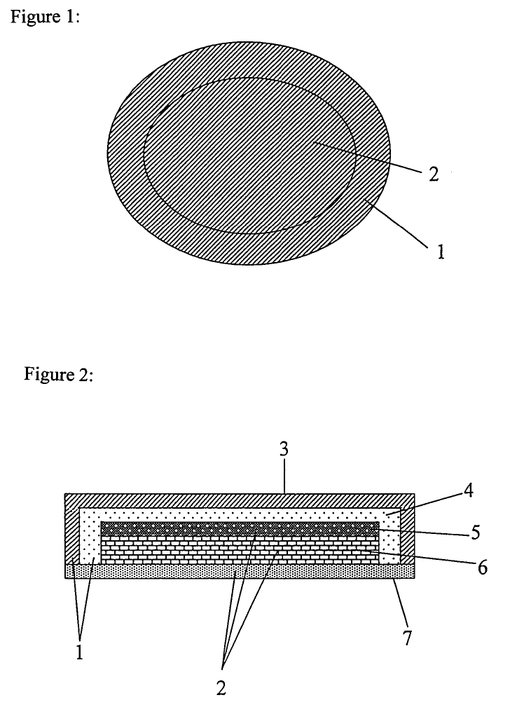

FIG. 1 shows a view from above onto the transdermal therapeutic system, wherein (1) denotes the “overtape” and (2) the rest of the system.

FIG. 2 shows a cross-section through the transdermal therapeutic system according to the invention having a self-adhesive matrix. The uppermost layer constitutes the active-ingredient-impermeable cover layer (3). Below that is an adhesive layer (4). Those two layers form the overtape (1). The next layer is again an active-ingredient-impermeable cover layer (5). The material of that cover layer (5) may be the same as or different from the material of the first cover layer (3). There then follows the self-adhesive matrix layer (6), which comprises the active ingredient and optional permeation inhibitors. The matrix-former is, in this case, the adhesive. A removable protective layer (7) forms a closure.

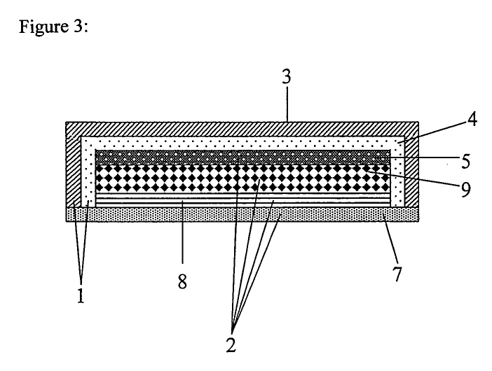

FIG. 3 shows a cross-section through the transdermal therapeutic system according to the invention having a non-self-adhesive matrix (9), which is provided with a separate adhesive layer (8).

The invention is illustrated in further detail by the Examples that follow, but without the scope of the invention being limited thereby.

### EXAMPLE 1

Composition of a self-adhesive matrix according to the invention for a TTS

| Constituents | Content in % by weight |
|---|---|
| Trandolapril diacid | 10 |
| Methanesulphonic acid | 2.4 |
| Aerosil ® 200 | 4 |
| Cetiol ® HE | 10 |
| Durotak ® 387-2353 | 73.6 |

The percentages by weight are based on the matrix weight.

Preparation Process:

Aerosil®, Cetiol® HE, adhesive (Durotak®) and ethyl acetate are weighed into a suitable stirred vessel (adhesive solution). In parallel thereto, trandolapril diacid and ethyl acetate are weighed into another suitable stirred vessel and homogenised; methanesulphonic acid is added and stirred until a clear solution has formed (active ingredient solution). The active ingredient solution is then added to the adhesive solution and homogenised. The mixture is applied to a film for the removable protective layer and dried in a drying channel. A PU film (e.g. Walotex 2204 ACK, 25 μm) for the active-ingredient-impermeable cover layer is then applied to the matrix. The patches are then cut out.

### EXAMPLE 2

Composition of a self-adhesive matrix according to the invention for a TTS

| Constituents | Content in % by weight |
|---|---|
| Ramipril diacid | 10 |
| Methanesulphonic acid | 2.5 |
| Aerosil ® 200 | 4.0 |
| Cetiol ® HE | 10 |
| Durotak ® 387-2510 | 73.5 |

The percentages by weight are based on the matrix weight.

Preparation Process:

Aerosil®, Cetiol® HE, adhesive (Durotak®) and ethyl acetate are weighed into a suitable stirred vessel (adhesive solution). In parallel thereto, ramipril diacid and ethyl acetate are weighed into another suitable stirred vessel and homogenised; methanesulphonic acid is added and stirred until a clear solution has formed (active ingredient solution). The active ingredient solution is then added to the adhesive solution and homogenised. The mixture is applied to the removable protective layer and dried in a drying channel. Further processing is carried out in the same manner as in Example 1.

### EXAMPLE 3

Composition of a self-adhesive matrix according to the invention for a TTS

| Constituents | Content in % by weight |
|---|---|
| Trandolapril diacid | 10 |
| Sodium hydroxide | 1 |
| Aerosil ® 200 | 4 |
| Cetiol ® HE | 10 |
| Durotak ® 87-4098 | 75 |

The percentages by weight are based on the matrix weight.

Preparation Process:

Aerosil®, Cetiol® HE and ethyl acetate are weighed into a suitable stirred vessel and stirred until a homogeneous suspension has formed. The adhesive Durotak® is weighed in (=adhesive solution) and the latter is homogenised using a suitable dispersing apparatus and is mixed overnight using a tumble roller. In parallel thereto, trandolapril diacid, sodium hydroxide and ethyl acetate are weighed into another suitable stirred vessel and stirred until a clear solution has formed (active ingredient solution). The active ingredient solution is then added to the adhesive solution and homogenised. The mixture is applied to a film for the removable protective layer and dried in a drying channel. Further processing is carried out in the same manner as in Example 1.

### EXAMPLE 4

Composition of a self-adhesive matrix according to the invention for a TTS

| Constituents | Content in % by weight |
|---|---|
| Ramipril diacid | 10 |
| Sodium hydroxide | 1 |
| Aerosil ® 200 | 4 |
| Cetiol ® HE | 10 |
| Durotak ® 87-4098 | 75 |

The percentages by weight are based on the matrix weight.

Preparation Process:

Aerosil®, Cetiol® HE and ethyl acetate are weighed into a suitable stirred vessel and stirred until a homogeneous suspension has formed. The adhesive Durotak® is weighed in (=adhesive solution) and the latter is homogenised using a suitable dispersing apparatus and is mixed overnight using a tumble roller. In parallel thereto, ramipril diacid, sodium hydroxide and ethyl acetate are weighed into another suitable stirred vessel and stirred until a clear solution has formed (active ingredient solution). The active ingredient solution is then added to the adhesive solution and homogenised. The mixture is applied to a film for the removable protective layer and dried in a drying channel. Further processing is carried out in the same manner as in Example 1.

### EXAMPLE 5

Composition of a self-adhesive matrix according to the invention for a TTS

| Constituents | Content in % by weight |
|---|---|
| Trandolapril ethyl ester | 10 |
| Aerosil ® 200 | 4 |
| Cetiol ® HE | 10 |
| Durotak ® 387-2510 | 76 |

The percentages by weight are based on the matrix weight.

Preparation Process:

Aerosil®, Cetiol® HE, trandolapril ethyl ester and ethyl acetate are weighed into a suitable stirred vessel (active ingredient solution). The active ingredient solution is then added to the adhesive solution (Durotak®) and homogenised. The mixture is applied to the removable protective layer and dried in a drying channel. Further processing is carried out in the same manner as in Example 1.

### EXAMPLE 6

Composition of a self-adhesive matrix according to the invention for a TTS

| Constituents | Content in % by weight |
|---|---|
| Ramipril ethyl ester | 10 |
| Aerosil ® 200 | 4 |
| Cetiol ® HE | 10 |
| Durotak ® 387-2510 | 76 |

The percentages by weight are based on the matrix weight.

Preparation Process:

Aerosil®, Cetiol® HE, ramipril ethyl ester and ethyl ac tate are weighed into a suitable stirred vessel (active ingredient solution). The active ingredient solution is then added to the adhesive solution (Durotak®) and homogenised. The mixture is applied to the removable protective layer and dried in a drying channel. Further processing is carried out in the same manner as in Example 1.

1. Matrix-controlled transdermal therapeutic system comprising (i) an active-ingredient-impermeable cover layer, (ii) a self-adhesive matrix layer, or a plurality of matrix layers of which at least the matrix layer exposed while applying the system is self-adhesive, or one or more matrix layers whose surface remote from the cover layer and intended for adhesion at the application site is coated with an adhesive, the matrix layers comprising at least one ACE inhibitor (angiotensin converting enzyme inhibitor) in the form of a dicarboxylic acid, which is derivatised to form a derivative selected from the following group:
diester,
di-salt obtainable with base(s) and
mono-salt obtainable with acid(s),
and (iii) a removable protective layer.

2. System according to claim 1, characterised by at least one ACE inhibitor from the group imidapril, fosinopril, moexipril, perindopril, ramipril, spirapril, cilazapril, benazepril and/or trandolapril in the form of a dicarboxylic acid, which is derivatised to form a diester, a di-salt formed with base(s) and/or a mono-salt formed with acid(s).

3. System according to claim 1, characterised by at least one ACE inhibitor from the group imidapril, fosinopril, moexipril, perindopril, ramipril, spirapril, cilazapril and/or trandolapril in the form of a dicarboxylic acid, which is derivatised to form a diester, a di-salt formed with base(s) and/or a mono-salt formed with acid(s).

4. System according to claim 2 and/or 3, characterised by ramipril and/or trandolapril, especially mono-sulphonic acid salt or disodium salt of trandolaprilat and/or ramiprilat.

5. Syst m according to claim 2 and/or 3, characteris d by an ethyl est r of trandolapril and/or ramipril.

6. System according to claim 1, 2 and/or 3, characterised in that the ACE inhibitor carries, in addition to a first ester group, a further ester group from the following group: m thyl, ethyl, propyl, isopropyl, butyl, tert-butyl, pentyl, hexyl, heptyl, octyl, nonane, decane groups and isomers thereof; the first ester group being freely selected, the first ester group being freely selected, the ACE inhibitor being a pharmaceutically acceptable compound, or the first and second ester group being identical.

7. System according to claim 6, characterised in that the further ester group is an ethyl group.

8. System according to at least one of the previous claims, characterised by a di-salt according to claim 1 which is obtainable using a base from the following group: sodium hydroxide, potassium hydroxide, magnesium hydroxide, calcium hydroxide, aluminium hydroxide, alkaline ammonium salt, organic amine, especially ethylenediamine, ethylamine, diethylamine, dipropylamine, diisopropylamine, tripropylamine, trihexylamine, tridodecylamine, dicyclohexylamine, ethanolamine, diethanolamine, triethanolamine, triisopropanolamine, 1-amino-2-propanol, 2-amino-2-methyl-1-propanol and oleylamine and heterocyclic amines, especially N-methylpiperazine and 1-(2-hydroxyethyl)pyrrolidine.

9. System according to claim 8, characterised by sodium hydroxide as base.

10. System according to at least one of claims 1 to 7, characterised by a mono-salt according to claim 1 which is obtainable using an acid from the following group: inorganic acid, especially hydrochloric acid, hydrobromic acid, hydriodic acid, nitric acid, sulphuric acid and phosphoric acid, organic carboxylic acid, especially salicylic acid, maleic acid, adipic acid, sorbic acid, malonic acid, 1,4-butanedioic acid, malic acid, pivalic acid, succinic acid, nicotinic acid, isonicotinic acid, furan-2-carboxylic acid, dichloroacetic acid and benzoic acid, fatty acids, especially lauric acid, myristic acid and oleic acid, aliphatic sulphonic acid, especially methane-, ethane-, propane-, isopropane-, butane-, isobutane-, pentane-, isopentane-, hexane-, heptane-, octane-, nonane-, decane-, undecane- and dodecane-sulphonic acid and aromatic sulphonic acid, especially toluene- and benzene-sulphonic acid.

11. System according to claim 10, characterised by methanesulphonic acid as acid.

12. System according to at least one of the previous claims, characterised in that ACE inhibitors,
(i) before formation of the di-salt thereof according to claim 1, together with base(s) for salt formation, in a molar ratio, separately from one another, or
(ii) before formation of a mono-salt according to claim 1, together with acid(s) for salt formation, in equimolar ratio, separately from one another, or
(iii) as the di-salt according to claim 1 or the mono-salt according to claim 1
have been incorporated into the system.

13. System according to at least one of the previous claims, characterised by a content of ACE inhibitor(s) of from 2 to 25% by weight and especially from 10 to 15% by weight, based on the matrix weight.

14. System according to at least one of the previous claims, characterised in that the system has, on that side of the cover layer which is remote from the matrix layer(s), (iv) a covering (overtape)
(i) which extends beyond the cover layer on all sides and which is provided with an adhesive that covers its surface or at least the region, in itself uninterrupted, extending beyond the cover layer, or
(ii) which covers over the surface of the cover layer but not does not extend beyond it and which is provided with an adhesive that covers its surface.

15. System according to claim 14, characterised in that the covering (overtape) provided with an adhesive completely covers over the active-ingredient-impermeable cover layer or is provided with one or more perforations above the cover layer or is of annular shape.

16. System according to at least one of claims 14 or 15, characterised in that the active-ingredient-impermeable cover layer and the covering provided with an adhesive are permeable to water vapour.

17. System according to one of the previous claims, characterised in that the active-ingredient-impermeable cover layer and the covering (overtape) provided with an adhesive are made from the same material.

18. System according to at least one of the previous claims, characterised in that the matrix layer(s) comprise(s) one or more permeation enhancers.

19. System according to claim 18, characterised by highly disperse silicon dioxide, polyoxyethylene 7-glycerol monococoate and/or 2-octyidodecanol (Eutanol G) as permeation enhancer(s).
